(19) United States
(12) Patent Application Publication
(10) Pub. No.: US 2021/0267794 A1
(43) Pub. Date: Sep. 2, 2021

(54) Miniaturized Intra-Body Controllable Cold Therapy Medical Devices and Methods

(71) Applicant: Miraki Innovation Think Tank, LLC, Cambridge, MA (US)

(72) Inventors: Christopher J. Velis, Lexington, MA (US); Matthew P. Palmer, Medford, MA (US)

(21) Appl. No.: 17/256,461

(22) PCT Filed: Jun. 24, 2019
(86) PCT No.: PCT/US19/38697

§ 371 Date: Dec. 28, 2020

Related U.S. Application Data
(60) Provisional application No. 62/691,870, filed on Jun. 29, 2018.

(51) Int. Cl. A61F 7/12

(52) U.S. Cl. CPC A61F 7/12; A61M 37/0015; A61F 2007/126

(57) ABSTRACT

A medical device for intra-body conveyance includes a host structure having a reservoir for containing a cold substance. The medical device includes at least one delivery apparatus for delivery of cold substance for administering cold therapy within a body.

### FIELD OF THE INVENTION

The present invention relates generally to a miniaturized intra-body controllable medical device. More specifically, the invention relates to the intra-body medical device having systems for providing cooling to regions within the body. Additionally, the device may include one or more of a propulsion system, a deployment system, a control system, a power supply system, an intra-device storage system, an imaging system, a therapy system, a sample and data gathering system, and/or a material dispensing system. Furthermore, the invention details materials for an intra-body controllable medical device, an interactive group of intra-body controllable medical devices, configurations for intra-body controllable medical devices, and methods of using intra-body controllable medical devices.

This disclosure relates to miniaturized intra-body controllable medical devices. Such devices may be externally controllable or may be fully autonomous. The devices may communicate via a tether or may communicate wirelessly. The devices may work independently or work together in a group.

### BACKGROUND OF THE INVENTION

Many medical procedures require the physician to gain access to regions within the body in order to complete a diagnosis or provide therapy to a patient. Often, physicians access internal regions of the body through the body's own natural orifices and lumens. Natural orifices include the nostrils, mouth, ear canals, nasolacrimal ducts, anus, urinary meatus, vagina, and nipples. The lumens include the interior of the gastrointestinal tract, the pathways of the bronchi in the lungs, the interior of the renal tubules and urinary collecting ducts, the pathways of the vagina, uterus, and fallopian tubes. From within these orifices and lumens, physicians can create an incision to gain access to almost any region of the body.

Traditional methods for gaining access to regions within the body include open surgical procedures, laparoscopic procedures and endoscopic procedures. Laparoscopic procedures allow the physician to use a small “key-hole” surgical opening and specially designed instruments to gain access to regions within the body. Initially, laparoscopic instruments were linear in nature, and required a straight obstruction free “line-of-sight” to access regions of the body. Endoscopic procedures allow the physician to access regions of the digestive system by passing flexible instruments through either the mouth or rectum.

Recently, physicians have begun to control these instruments using robots. These robots are typically connected in master/slave configuration, where the robot translates the physician's movements into instrument movements. Robotic controls have also allowed for advent of flexible laparoscopic instruments. Medical robots still require a physician to be actively controlling the movements and actions of the devices being controlled and require large expensive capital equipment and dedicated operating room spaces.

Additionally, pill capsules have been invented that allow for a patient to ingest the capsule and as it passes through the digestive system takes pictures. There are no means for: controlling the motion of these devices, tracking or controlling the orientation, speed or location of these devices, accurately knowing where pictures were taken, and performing any type of surgical procedure or delivering therapy.

Furthermore, it is known that cold temperatures can be used to preferentially kill or destroy fat cells. Currently, cold therapy for fat reduction utilizes applicators external to the body to deliver cold to the fat tissue through the skin. Current methods of application limit the regions of fat that can be targeted to those adjacent to the skin. Furthermore, procedures must be performed in offices to allow for the movement of the applicators to different regions.

Thus, improvements are desirable in this field of technology. It would be beneficial to combine the ability to deliver cold therapy with the maneuverability of miniaturized intra-body controllable medical device including capsule systems or other structures. It would be beneficial to provide a means for delivering targeted cold therapy to regions of the body both adjacent to and not adjacent to the skin.

Further, it would be beneficial to combine the ability to perform surgical procedures and provide therapy using robotic instruments with the footprint, size, and maneuverability of miniaturized intra-body controllable medical devices to provide a means for controlling the movement of a medical device so that the surgeon can navigate it to a specific location for treatments such as cold treatments.

### SUMMARY

There is disclosed herein a medical device for intra-body conveyance that is directed to administering cold therapy within a body (e.g., a human body). The medical device includes a host structure that has a reservoir for containing a cold substance (e.g., a liquid, an aqueous solution, a plurality of particulate matter, an isotonic solution, a saline solution, a gel, a slurry, a fat destroying substance and a vascoconstrictor). The medical device includes one or more delivery apparatuses (e.g., a needle) in communication with the host structure, for delivery the cold substance for administering cold therapy within a body.

In some embodiments, the delivery apparatus or needle is configured to inject the cold fluid inside the body.

In other embodiments, the delivery apparatus or needle is configured to circulate the cold substance to a predetermined cold therapy receiving region and returns the cold substance to the host structure for further cooling.

In certain embodiments, the delivery apparatus or needle is a microneedle having a diameter of less than about 1.0 mm.

In particular embodiments, the medical device is in communication with at least one repository. The at least one repository includes at least one of a heat sink, a heat exchanger, a chemical reactor and a storage vessel.

In some embodiments, host structure includes at least one of a clinically inert material, a sterilizable material, an elastomeric material, a chemically reactive material, a chemically inert material, a disintegrable material, a dissolvable material, a collapsible material and a material having physical and chemical properties to withstand exposure to bodily fluids for a predetermined period of time.

In another aspect of the present invention, a plurality of medical devices is in communication with at least one repository. The at least one repository includes at least one of a heat sink, a heat exchanger, a chemical reactor and a storage vessel. At least one the plurality of medical devices includes a host structure having a reservoir for containing a cold fluid and at least one needle for delivery of cold therapy within a body. The at least one repository can be positioned in at least one of inside the body or outside the body.

In one embodiment of this aspect, the at least one repository is connected to at least one of the plurality of medical devices by at least one of a network of conduits, tubes, cannulas, capillaries, heat conducting materials and ducts.

In another aspect, a method for using the medical device is directed to using a cold fluid to effect destruction of fat cells with a body.

There is further disclosed herein a method for using a medical devices for administering cold therapy in a body. The method includes disposing the medical device inside the body, proximate a cold therapy receiving site. A cold substance is discharged or circulated into the receiving site for a predetermined time, thereby administering the cold therapy within the body.

In certain embodiments, the method of using the medical devices is directed to use for treatment of scars, wrinkles, disorders of pigmentation, hyperhidrosis, destroying subcutaneous fat cells, destroying visceral fat cells, inducing collagen synthesis and/or inducing hair restoration.

In certain embodiments, a method of providing therapeutic treatment to a patient includes inserting a medical device into a patient's body lumen; navigating the medical device to a specific site in need of a site-specific cold therapy; and delivering the site-specific cold therapy in proximity to the site in need of the therapy.

### DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1A illustrates a representative intra-body controllable medical device formed in accordance with the present invention;

FIG. 2 illustrates an alternative representation of an intra-body controllable medical device formed in accordance with the present invention;

FIG. 3 illustrates an interactive group of intra-body medical devices for delivering cold therapy to regions within a human body; and

FIG. 4 illustrates a representative intra-body controllable medical device configured for the delivery of cold therapy using micro needles.

### DETAILED DESCRIPTION OF THE PREFERRED EMBODIMENT

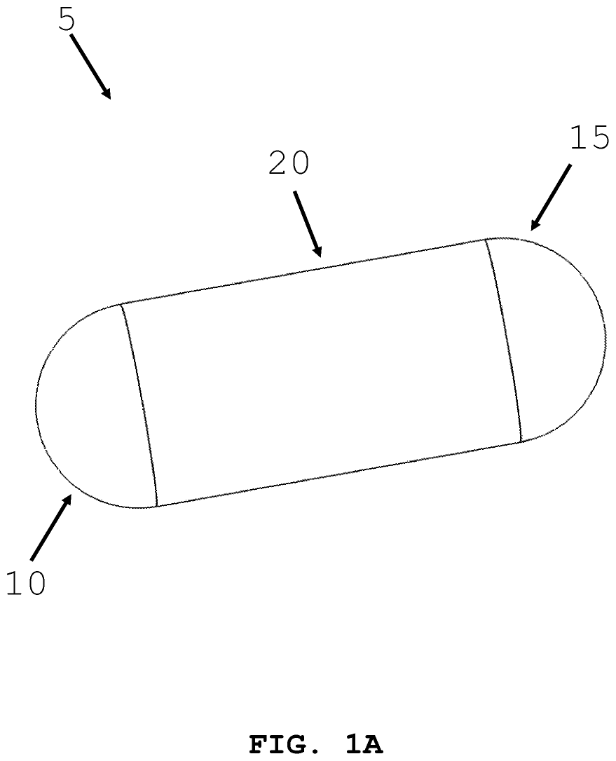

FIG. 1A illustrates an exemplary intra-body controllable medical device (hereinafter “the medical devices”). In one embodiment, the intra-body controllable medical device 5 is capsule shaped. Intra-body controllable medical device 5 has a distal end 10, a proximal end 15, and body 20 connecting the distal end 10 and proximal end 15. A control unit, a power supply system, an intra-device storage system, an imaging system, a therapy system, a sample and data gathering system, and a material dispensing system may be located within body 20 of the medical device 5, as described herein.

The intra-body controllable medical device may be sized according to the anatomy that it will need to navigate, and the method used to deliver it. For example, overall dimensions for an intra-body controllable device operating within the gastrointestinal track may have a diameter of about 25 mm and a length of about 75 mm. More preferably, the device may have a diameter of about 15 mm and a length of about 50 mm. Most preferably, the diameter may be less than about 15 mm and a length of less than about 50 mm.

Overall dimensions for an intra-body controllable device that is delivered using a scope may have a diameter of about 20 mm in diameter and a length of about 75 mm. More preferably, the diameter may be about 15 mm and the length may be about 50 mm. Most preferably, the diameter may be less than 15 mm and the length less than 50 mm. Control system, power supply system, intra-device storage system, imaging system, therapy system, sample and data gathering system, and material dispensing systems are sized to fit within these dimensional guidelines.

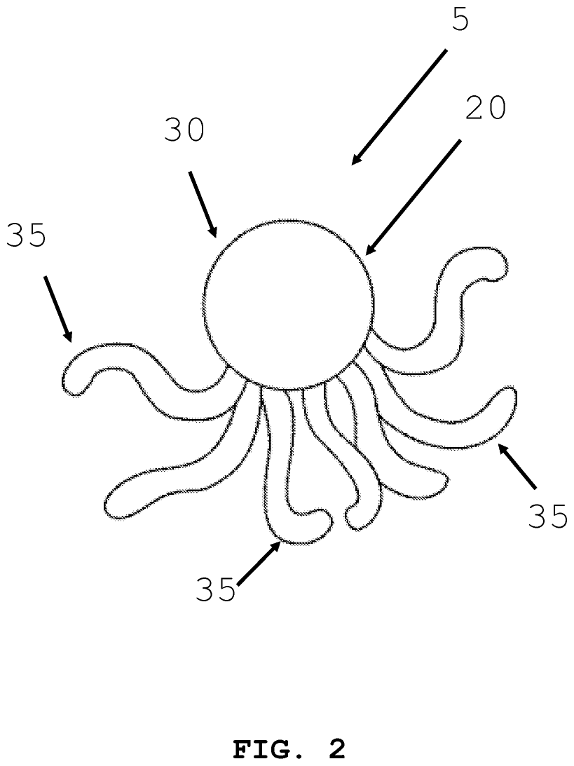

As shown in the exemplary embodiment of FIG. 2, the intra-body controllable medical device 5 may be octopus shaped. The intra-body controllable medical device has a main body 30, and appendages 35. Appendages 35 may be used for propulsion, covering or wrapping the host structure 20, forming a portion of the host structure 20 or to perform a therapeutic or diagnostic task. A control unit, power supply systems, an intra-device storage system, an imaging system, a therapy system, a sample and data gathering system, and a material dispensing system similar to those shown and described with reference to FIG. 1B, may be located within main body 30 and/or appendages 35 of the device or in the interior areas of the host structure 20.

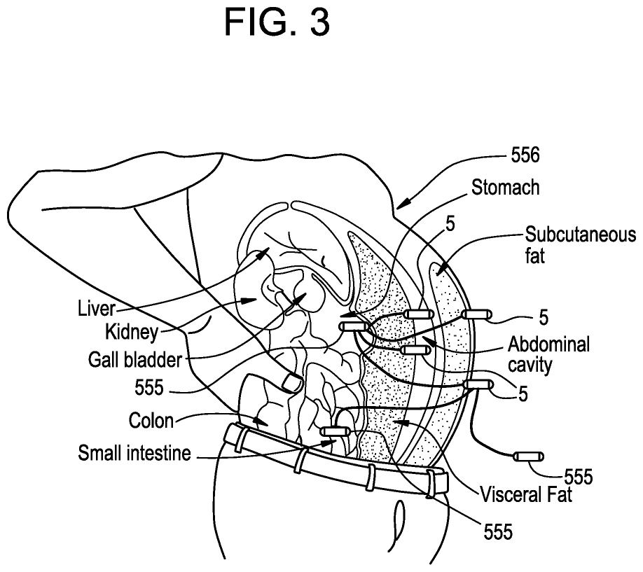

As shown in FIG. 3, intra-body medical device 5 or an interactive group thereof may be used to deliver cold therapy within a patient. A plurality of medical devices may be in communication with one or more repositories 555. The repositories 555 can include heat sinks, heat exchangers, chemical reactors and/or storage vessels. The plurality of medical devices has a cooling system and/or a material delivery or discharge system disposed therein or thereon. The repositories are positioned inside a human body 556 and/or outside the human body. The intra-body medical devices 5 can be connected to each other and the repositories 555 by a network of conduits (e.g., tubes, cannulas, capillaries, heat conducting materials and/or ducts).

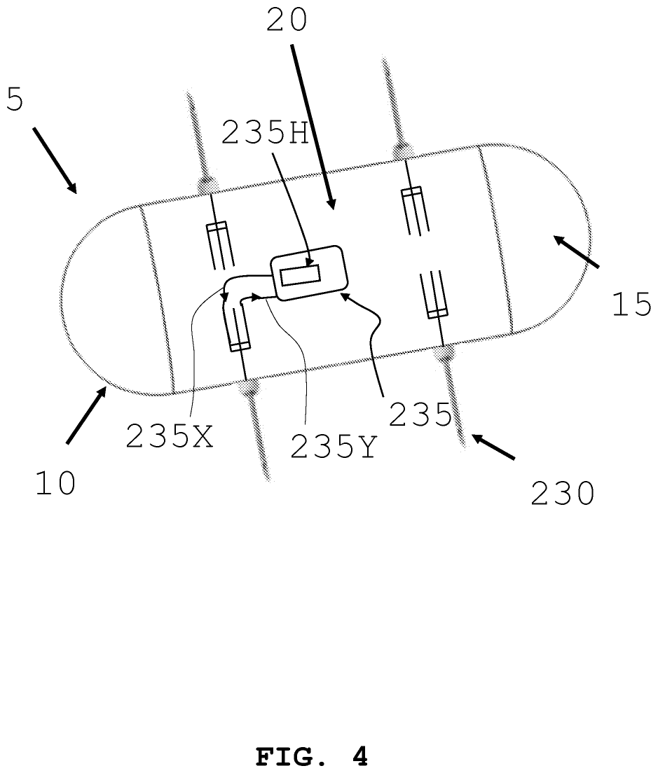

As shown in FIG. 4, intra-body medical device 5 can include a miniature discharge apparatus such as a needle 230 attached to host structure 20 includes an interior area 20A having a reservoir 235. The needle 230 may be attached to the reservoir 235. The reservoir may contain a cold substance such as a liquid, an aqueous solution, a plurality of particulate matter, an isotonic solution, a saline solution, a gel, a slurry, a fat destroying substance and a vascoconstrictor, that can be injected through needle 230 into the body, for example, in the proximity into fat cells (e.g., subcutaneous fat cells and/or visceral fat cells) or other cold therapy receiving region, in order to destroy the fat or otherwise administer the cold therapy. As shown in FIG. 4, the reservoir 235 includes a heat exchanger/heat sink 235H therein. The cold substance is discharged from the heat exchanger/heat sink 235H via a discharge line 235X that is in communication with the discharge device (e.g., needle) 230 for circulating or discharging the cold substance therefrom. Alternatively, the needle may be closed and instead of injecting the cold substance (e.g., cold fluid or slurry) into the fat or other cold therapy receiving region, the cold slurry or fluid may be circulated, in a closed loop manner, through the needle to generate localized cooling in the cold therapy receiving region. For example, as shown in FIG. 4, the cold substance is returned to the heat exchanger/heat sink 235H in the reservoir 235 disposed in the host structure 20, via a return line 235Y, for further cooling in the heat exchanger/heat sink 235H.

The needle 230 may be sized so as not to create scars. Preferably the needle is smaller than a 16-gauge needle. Most preferable the needle may be a “micro” needle—with a diameter less than 1.0 mm. In addition to being able to provide cooling through the needle, micro needle may create small holes known as micro-conduits that generate minimal damage to the epidermis. This process can lead to the generation of growth factors which stimulate the production of collagen and elastin in the papillary layer of the dermis. These micro-conduits may be used to treat scarring and wrinkles, enable skin rejuvenation and brightening, improve the appearance of skin (anti-ageing), treat disorders of pigmentation, hyperhidrosis, striae, induce collagen synthesis under the epidermis, treat hair pathology as it may stimulate stem cells in the dermal papilla, increase blood flow to hair follicles, and recruit growth factors and signaling pathways which induce hair restoration, and fill in fine lines and plump the skin.

Alternatively, the heat exchanger/heat sink 235 may be used to cool the surface of intra-body controllable medical device 5.

The present invention is directed to configurations for intra-body controllable medical devices and in particular to disposable, disintegrable and selectively collapsible intra-body controllable medical devices and materials and structures thereof. The intra-body controllable medical devices can be manufactured of a material such as an elastomer (e.g., nitrile) that can expand and contract, for example, by inflating and deflating them. The intra-body controllable medical devices can be manufactured from a biodegradable, disintegrable or dissolvable material, including paper, starches, biodegradable material such as gelatin or collagen and/or synthetic natural polymers. The collapsible intra-body controllable medical devices can be configured to be flattened, extruded, stretched or disassembled in the lumen. Thus, the intra-body controllable medical devices can be disposed of in the lumen or via discharge therefrom without the need to recover the intra-body controllable medical devices for analysis, inspection or future use.

Although the present invention has been disclosed and described with reference to certain embodiments thereof, it should be noted that other variations and modifications may be made, and it is intended that the following claims cover the variations and modifications within the true scope of the invention.

1. A medical device for intra-body conveyance, the medical device comprising:
a host structure comprising a reservoir for containing a cold substance; and
at least one delivery apparatus in communication with the host structure for delivery of the cold substance to regions within a body for administering cold therapy in the body.

2. The medical device of claim 1, wherein the at least one delivery apparatus comprises at least one needle configured to inject the cold substance into the body.

3. The medical device of claim 2, wherein the delivery apparatus circulates the cold substance to a predetermined cold therapy receiving region and returns the cold substance to the host structure for further cooling.

4. The medical device of claim 2, wherein said at least one needle is a microneedle having a diameter of less than about 1.0 mm.

5. The medical device of claim 1, wherein said cold substance is at least one of a liquid, an aqueous solution, a plurality of particulate matter, an isotonic solution, a saline solution, a gel, a slurry, a fat destroying substance or a vascoconstrictor.

6. The medical device of claim 1, wherein the medical device is in communication with at least one repository, the at least one repository comprising at least one of a heat sink, a chemical reactor or a storage vessel.

7. The medical device of claim 1, wherein the host structure comprises at least one of a clinically inert material, a sterilizable material, an elastomeric material, a chemically reactive material, a chemically inert material, a disintegrable material, a dissolvable material, a collapsible material or a material having physical and chemical properties to withstand exposure to bodily fluids for a predetermined period of time.

8. A plurality of medical devices in communication with at least one repository, the at least one repository including a least one of a heat sink, a heat exchanger, a chemical reactor or a storage vessel, at least one of the plurality of medical devices comprising a host structure having a reservoir for containing a cold substance and at least one needle for delivery of cold substance for administering cold therapy within a body, wherein the at least one repository is positioned in at least one of inside the body or outside the body.

9. The plurality of medical devices of claim 8, wherein the at least one repository is connected to at least one of the plurality of medical devices by at least one of network of conduits, tubes, cannulas, capillaries, heat conducting materials or ducts.

10. A method for using the medical device of claim 1, the method comprising:
disposing the medical device inside the body proximate a cold therapy receiving region;
discharging the cold substance into the region for a predetermined time; and
administering the cold therapy to the region inside the body.

11. A method for using the medical device of claim 1, the method being directed to use for treatment of at least one of scars, wrinkles, disorders of pigmentation, hyperhidrosis, destroying subcutaneous fat cells, destroying visceral fat cells, inducing collagen synthesis or inducing hair restoration.

12. A method of providing therapeutic treatment to a patient comprising:
inserting a medical device into a patient's body lumen;
navigating the medical device to a specific site in need of a site-specific cold therapy; and
delivering the site-specific cold therapy in proximity to the site in need of the therapy.
